(19) United States
(12) Patent Application Publication
(10) Pub. No.: US 2012/0130162 A1
(43) Pub. Date: May 24, 2012

(54) ENDOSCOPE SYSTEM

(75) Inventors: Martin DOLT, Knittlingen (DE); Martin WEIGEL, Maulbronn (DE)

(73) Assignee: RICHARD WOLF GMBH, Knittlingen (DE)

(21) Appl. No.: 13/296,281

(22) Filed: Nov. 15, 2011

(30) Foreign Application Priority Data
Nov. 18, 2010 (DE) 10 2010 051 797.6

(51) Int. Cl. A61B 1/06

(52) U.S. Cl. 600/104

(57) ABSTRACT

An endoscope system has imaging optics and includes at least one adjustable optical element. A working instrument is positionable relative to the imaging optics. A displacement measuring device is designed for detecting a distance covered by the working instrument with its positioning. A control device adjusts the optical element in dependence on a detected displacement.

### BACKGROUND OF THE INVENTION

The imaging optics of medical and technical endoscopes is usually preset to an operating distance which is adapted to the application purpose of the endoscope. A certain depth of field, i.e., a limited region which extends along the optical axis by the envisaged working distance and which may be imaged in a focused manner, results depending on the technical constraints of the imaging optics (such as for example depending on their maximum dimensions, on the brightness of the optical image as well as on the settings of the topography of the objects to be examined). However, objects which are distanced significantly different from the preset working distance and in particular moved objects must often be imaged in a focused manner. Such objects, for example, are moved working instruments of endoscope systems, for example, medical working instruments for examining and manipulating tissue.

For this, it is known to change the distance of the focal plane of the imaging optics, for example, by way of manual refocusing or however by way of dimming the imaging optics to such an extent, that the depth of field is increased. Such refocusing or dimming methods are also known in an automated manner as autofocusing or autoiris methods respectively.

However, the disadvantage is the fact that the refocusing ability of imaging optics is often limited. Autofocusing methods moreover, with the imaging of structurally weak tissue surfaces, however, are problematic due to suddenly occurring sharpness corrections. Moreover, rapid changes of the working distance often entail interfering sharpness corrections which easily irritate the user. Moreover, objects additionally moved into the picture may lead to fluctuations of the focal plane between these objects and the picture background. The dimming for increasing the depth of field on the other hand often requires such a great dimming, that the faintness of the optical image which this entails greatly limits the work with the endoscope or even rules this out. Moreover, the optical resolution of the endoscope system reduces due to the large dimming with the dimming or autoiris method.

### BRIEF SUMMARY OF THE INVENTION

Against the above background, it is an objective of a preferred embodiment of the present invention to create an endoscope system with which moving objects may be sharply imaged in a reliable and user-friendly manner.

The above objective is achieved by an endoscope system with the described and shown. The endoscope system according to a preferred embodiment of the present invention includes imaging optics which comprise at least one adjustable optical element. Moreover, the endoscope system includes a working instrument positionable relative to the imaging optics, a displacement measuring device which is designed for detecting the distance covered by the working instrument with its positioning, and a control device which adjusts the optical instrument in dependence on the detected displacement. A movement of the working instrument relative to the imaging optics is thereby to be understood as a movement of the working instrument relative to a stationary part of the imaging optics, for example, relative to at least one picture sensor or an eyepiece of the imaging optics. With the endoscope system according to a preferred embodiment of the present invention, the position of the focal plane of the imaging optics, i.e., of that plane which contains objects sharply imaged by the imaging optics, and which is desired at the respective point in time, is determined by the positioning of the working instrument relative to the imaging optics. For example, the working instrument is a medical instrument for the examination, removal or for the manipulation of tissue, which is imaged by way of imaging optics, so that the guidance and/or the working manner of the working instrument may be tested and controlled by way of visual control by way of imaging optics. The displacement measuring device of the endoscope system according to a preferred embodiment of the present invention thereby detects the distance covered by the working instrument with the positioning of this, in particular the displacement along the optical axis. Thus the displacement measuring device always detects the distance changes of the focal plane of the imaging optics, which are necessary for ensuring a sharp or focused image of the working instrument. The settable optical element of the imaging optics, by way of the control device, is now set in dependence on the detected displacement, in a manner such that the focal plane of the imaging optics corresponds to the distance of the working instrument to the imaging optics, i.e., the optical element is thus set for focusing or refocusing of the optical image of the working instrument by way of the imaging optics.

The setting of the settable optical element is preferably effected in a manner such that the distance of the focal plane of the imaging optics is just about adjusted to the distance of the working instrument to the imaging optics. Distance of the focal plane in the contents of a preferred embodiment of the present invention is thereby always to be understood as the distance of the focal plane to the object-side or distal end of the imaging optics, and thus represents a characteristic of the imaging optics themselves. In an alternative further formation of the present invention, at least one distal part of the imaging optics, in particular a distal front lens, may be set with regard to its position along the optical axis, for example in a rigid endoscope. The distal part of the imaging optics, in this further formation of the present invention, may be displaced relative to the proximal optical elements of the imaging optics without a noticeable change of the optical image. In this case, preferably the distance of the distal part of the imaging optics to the working instrument is adapted to the working distance or the distance of the focal plane of the imaging optics. In both further formations mentioned above, the settable optical element is thus always set such that the actual distance of the working instrument to the imaging optics and the distance of the focal plane of the imaging optics are adjusted to one another.

In this manner, with the endoscope system according to a preferred embodiment of the present invention, the working instrument may always be imaged in a sharp manner with the imaging optics. Advantageously, with the endoscope system according to a preferred embodiment of the present invention, with the distance covered by the working instrument, an operating parameter of the endoscope system itself is used, and not just passively recorded information requiring interpretation, for example such as the picture information with autofocusing solutions. In this manner, the endoscope system according to a preferred embodiment of the present invention, differently to the autofocusing solutions, does not have to be based solely on the imaging of contrast-rich objects, but even permits a sharp imaging with structurally weak or contrast-weak objects, or with dull conditions. Suddenly occurring sharpness corrections do not occur with the endoscope system according to a preferred embodiment of the present invention, so that the user may operate free of irritation. Moreover, with the endoscope system according to a preferred embodiment of the present invention, it is not necessary to enlarge the depth of field by way of dimming. Thus, the brightness as well as the resolution of the optical image is ensured, so that the working instrument and objects close to this may be imaged in a bright and at the same time adequately sharp manner, even with dim conditions.

Preferably, with the endoscope system according to a preferred embodiment of the present invention, the imaging optics are arranged with the working instrument in a common shank. Advantageously, the working instrument is arranged in the shank in a positionable manner along the shank axis, wherein the imaging optics at least partly is arranged in a stationary manner to the shank. In this manner, the positioning of the working instrument relative to the imaging optics is fixed to a single direction. The position of the working instrument is thus particularly easy to detect. Usefully, the working instrument is displaceable along an axis which coincides with the optical axis of the imaging optics or runs parallel to this and in particular is offset slightly—compared to typical working distances—to the optical axis of the imaging optics. Particularly advantageously, the optical axis of the imaging optics runs parallel to the shank axis, wherein the working instrument is displaceable along the shank axis.

Preferably, with the endoscope system according to a preferred embodiment of the present invention, the control device for setting the optical element is designed in a manner such that the distance of the focal plane or the position of the imaging optics is changed relative to the detected displacement of the working instrument along the optical axis. A sharp image of the working instrument is always ensured with this.

Preferably, with the endoscope system according to a preferred embodiment of the present invention, the control device is designed for adjusting the optical element in a manner such that the focal plane of the imaging optics adjusts synchronous with the working instrument. Alternatively, the control device may also adjust the position of a distal part of the imaging optics, in a manner such that this distal part, for example a front lens of the imaging optics, synchronously co-moves with the working instrument along a common axis.

In a further preferred formation of the present invention, the working instrument is displaceable obliquely to the optical axis of the imaging optics, i.e., the imaging optics and the working instrument are arranged with an angular offset to one another. For example, the axis, along which the working instrument is displaceable, and the optical axis of the imaging optics (at least during operation) of the endoscope system are arranged to one another in a stationary manner at a known angle. Usefully, by way of adjusting the optical element by way of the control device, the distance of the focal plane or the position of a distal part of the imaging optics in this further formation is always changed by the displacement of the working instrument along the optical axis. This means that the distance of the focal plane is not changed by the same amount, by which the working instrument displaces, but rather the angular offset between the optical axis and the movement axis of the working instrument is taken into account and the distance of the focusing plane is adjusted by such an amount, that the distal end of the working instrument, i.e., the working region of the working instrument, lies in the focal plane of the imaging optics.

Preferably, with the endoscope system according to a preferred embodiment of the present invention, the at least one adjustable optical element is an electromechanically and/or piezoelectrically displaceable and/or electrically changeable element, in particular a liquid lens, whose focal width may be electrically changed.

Preferably, the working instrument is or includes at least one of the following elements: a HF-electrode, in particular a HF-resection electrode, a cutting loop, a stone loop, a laser fiber, a lithotripsy electrode, gripper forceps, a motor-driven working instrument, in particular a cutter or a measurement probe. It is indeed with the previously mentioned working instruments the case of working instruments which are regularly monitored via imaging optics, so that a continuously sharp image by way of the imaging optics of the endoscope system is particularly advantageous here.

Particularly preferably, with the endoscope system, the displacement measuring device is designed for the contact-free detection of the distance covered by the working instrument. Advantageously thereby, the working instrument and parts of the displacement measuring device of the endoscope shank system may be encapsulated independently of one another, so that a particularly sealed design of the endoscope system may be realized. For example, a part of the encapsulation may separate parts of the displacement measuring device from one another or from the working instrument, for example a displacement transmitter and a displacement pick-up, wherein transmitter and pick-up are to be understood in their broadest sense, to the effect that transmitter is to be understood as a sensor element which affects the pick-up which is to be understood as a sensor element associated with the transmitter.

Particularly preferably, with the endoscope system according to a preferred embodiment of the present invention, the displacement measuring device includes a displacement pick-up which is arranged in a fixed manner with regard to the imaging optics, and a displacement transmitter which corresponds to the displacement pick-up and which is arranged in a fixed manner with regard to the working instrument. In an alternative preferred further formation, with the endoscope system, the displacement measuring device includes a displacement pick-up which is arranged fixed or firmly to the working instrument, and a displacement transmitter which corresponds to the displacement pick-up and is arranged fixed or firmly to the imaging optics. In these further formations of the present invention, the position of the working instrument relative to the imaging optics and/or a further part which does not co-move with the working instrument at the same time, may be detected in a simple manner.

Usefully, the displacement measuring device includes at least one magnet encoder and/or magnetic field sensors and/or a plunger coil. For example, a magnet encoder is a displacement transmitter of the displacement measuring device, and a magnetic field sensor is a displacement pick-up corresponding to the magnet encoder. For example, with regard to the displacement measuring device, a component of ferromagnetic material may also form the displacement transmitter which, with a relative movement of the working instrument and imaging optics with a variable depth corresponding to the relative positioning of the working instrument and imaging optics, projects into a plunger coil. The plunger coil thereby forms a displacement pick-up, whose inductance is changed by the projecting depth of the ferromagnetic component. Plunger coils are thereby in particular suitable for detecting relative larger relative movements of the working instrument and imaging optics, for example with flexible endoscope systems.

Suitably, with regard to the endoscope system according to a preferred embodiment of the present invention, at least one of the following components may be prepositioned: working instrument, displacement pick-up and/or displacement transmitter. For example, with a change of the working instrument or the imaging optics of the endoscope system, an initially envisaged distance to the working instrument and the imaging optics may be changed. For example, a change of such parameters may also occur with an exchange of displacement pick-up and/or displacement transmitter. In the above described further formation of the present invention, the working instrument, displacement pick-up and/or displacement transmitter may then be manually prepositioned to one another into a relative position envisaged for operation of the endoscope system. It is then starting from this initial position that the displacement detection of the working instrument and the corresponding refocusing via the adjustable optical element is effected.

In an alternative, preferred further formation of the present invention, with the endoscope system according to the present invention, the control device is designed for setting the optical element in dependence on an initial positioning of the working instrument and/or displacement pick-up and/or displacement transmitter. In this case, deviations of an initial position of the working instrument and imaging optics from an initially provided arrangement for operation of the endoscope system may be taken into account by the control device, by way of a correcting adaptation of the control variables for setting the optical element. Changes in the arrangement, for example after the exchange of the working instrument and imaging optics, and changes of the displacement pick-up relative to the displacement transmitter may be accordingly corrected.

The working instrument according to a preferred embodiment of the present invention is designed for incorporation into an endoscope system according to the present invention as described above and is provided with a displacement pick-up and/or displacement transmitter. The working instrument thereby includes a displacement pick-up which corresponds to a displacement transmitter of the endoscope system according to a preferred embodiment of the present invention, or, however, includes such a displacement transmitter which corresponds to a displacement pick-up of the endoscope system according to the present invention. The working instrument according to a preferred embodiment of the present invention thereby indeed forms such a working instrument of the endoscope system according to the present invention, which cooperates with the imaging optics for refocusing, as previously described. With regard to the working instrument according to a preferred embodiment of the present invention, it is the case for example of a HF-resection electrode, a stone loop, a cutter loop, a working instrument with a laser fiber, lithotripsy probe, gripper forceps, HF electrode, a working instrument with a laser fiber, a cutter or a measurement probe. For example, the working instrument may be envisaged as an exchangeable part for an endoscope system according to a preferred embodiment of the present invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is an endoscope system according to a preferred embodiment of the present invention, with a rigid shank, in a basic sketch;

FIG. 2 is an endoscope system according to another preferred embodiment of the present invention, with a rigid shank and a distally arranged picture sensor, in a basic sketch;

FIG. 3 is an endoscope system according to another preferred embodiment of the present invention, with a flexible shank, in a basic sketch;

FIG. 4 shows imaging optics of the endoscope system according to FIG. 2, in a basic sketch;

FIG. 5 shows the imaging optics according to FIG. 4, with a changed distance of the working instrument to the imaging optics; and

FIG. 6 shows imaging optics with an optical axis which is angled compared to the working instrument.

### DETAILED DESCRIPTION OF THE INVENTION

Certain terminology is used in the following description for convenience only and is not limiting. The word “front” designates a direction in the drawings to which reference is made. Unless specifically set forth herein, the terms “a,” “an” and “the” are not limited to one element, but instead should be read as meaning “at least one.” The terminology includes the words noted above, derivatives thereof and words of similar import.

The endoscope system 5 represented in FIG. 1 is a rod-lens endoscope system and preferably includes a working instrument in the form of a loop 10. For the visual control, the endoscope system 5 preferably includes an endoscope shank 15 with imaging optics. In this preferred embodiment example, the endoscope shank 15 is a urological resection shank and is designed in a rigid manner. The loop 10 is movable along the optical axis 20 of the imaging optics of the endoscope shank 15 and is led via an instrument shank 25 which extends along the endoscope shank 15 and is held on this.

In the usual manner, an actuation means 30 for the axial movement of the loop 10 is arranged at the proximal end region of the endoscope shank 15. The endoscope shank 15 proximally of the actuation means 30 ends in an eyepiece coupling 35. The eyepiece coupling 35 couples the endoscope shank 15 on a removable camera head 40.

The movement of the loop 10 along the optical axis 20 is effected by way of the displacement of an actuator 45 which is displaceable in the instrument shank 25 and which is coupled in movement to the loop 10 and the actuation means 30. The axial displacement of the actuator 45 is detected by way of a displacement transmitter 50 which is arranged within the endoscope shank 15 and is arranged in a fixed manner with regard to the endoscope shank 15. A displacement pick-up 55 is provided on the actuation means 30 in a fixed or rigid manner with regard to the actuation means 30 and thus also in a fixed manner with regard to the actuator 45 and detects the relative displacement of the actuator 45 and thus of the loop 10 to the displacement transmitter 50.

The detected displacement of the loop 10 along the optical axis 20 is evaluated by way of a control device 60, to which the displacement pick-up 55 is signal connected. Depending on the axial position of the loop 10, the control device 60 delivers an electric control variable, with which a settable optical element within the camera head 40, here a liquid lens 65 with an electrically influencable focal width, may be set and controlled. For this, the control device 60 is connected to a control contact of the liquid lens 65. The liquid lens 65 is thereby set such that the image (not represented in the drawing) of the loop 10 on a sensor arranged in the camera head 40 is focused, i.e., the distance of the focal plane of the imaging optics of the endoscope system 5 is set to the distance of the loop 10 to the distal end of the imaging optics in the endoscope shank 15. If the distance of the loop 10 to the distal end of the imaging optics in the endoscope shank 15 changes, then the displacement of the loop 10 is detected via the displacement transmitter 50 and the displacement pick-up 55 and a new control variable for the liquid lens 65 is determined in the control device 60. After a renewed computation of the electric control variable for the liquid lens 65, the control unit transfers the control variable to the control contact of the liquid lens 65. As a result of this, the focal width of the liquid lens 65 is changed such that the distance of the focal plane of the imaging optics of the endoscope system 5 is adapted to the changed position of the loop 10.

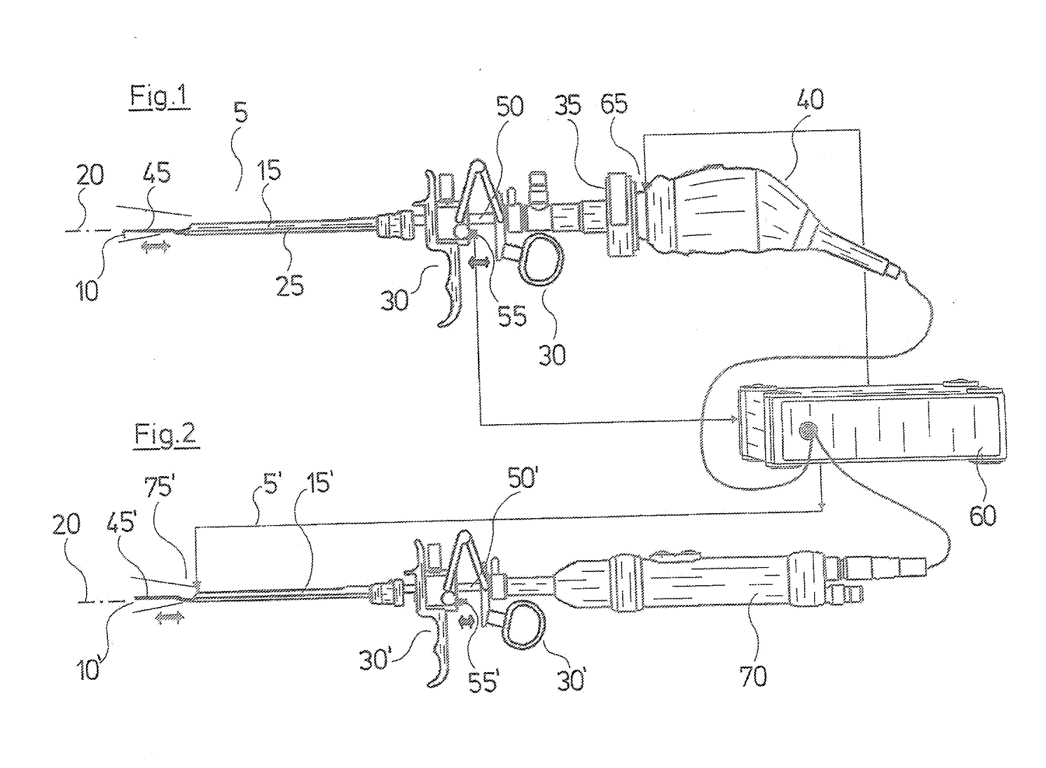

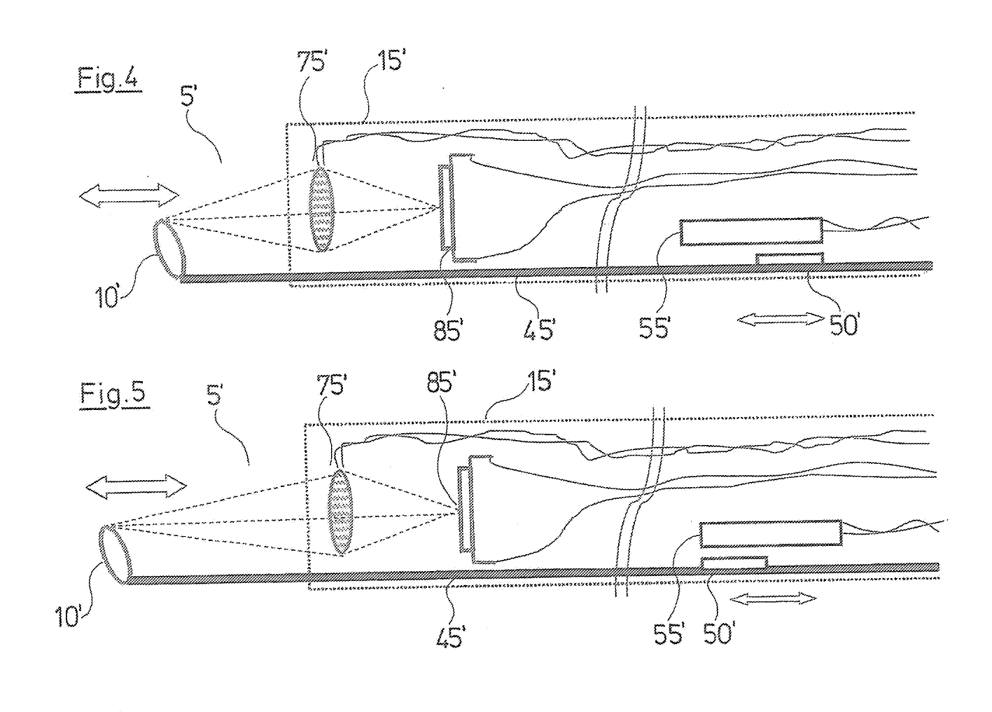

The endoscope system 5′ which is represented in FIGS. 2, 4 and 5 is likewise an endoscope system 5′ with a rigid endoscope shank 15′. The endoscope system 5′ is a sensor endoscope, i.e. the endoscope system has a picture sensor 85′ which is arranged in a distal region of the endoscope shank 15′ (FIGS. 4 and 5). With regard to the endoscope system 5′, the adjustable optical element is a liquid lens 75′ with an electrically influencable focal width which forms a front lens at the distal end of the endoscope shank 15′.

With the endoscope system 5′, the displacement of the loop 10′ along the optical axis 20 is also effected via a displacement transmitter 50′ and a displacement pick-up 55′. The displacement pick-up 55′ is hereby located in a fixed manner with regard to the imaging optics and to the endoscope shank 15′, within the endoscope shank 15′. The displacement transmitter 50′ thereby is arranged in a fixed or rigid manner with regard to the actuator 45′ and to the loop 10′, which are coupled in movement to the actuation means 30′. Electrical leads connected to a control device 60′ are connected onto the displacement pick-up 55′ and are led together with the electrical leads of the picture sensor 85′ and the liquid lens 75′, through the endoscope shank 15′. The liquid lens 75′ is always set by way of the control device 60′ in a manner depending on the axial displacement of the loop 10′ (see in particular FIGS. 4 and 5) detected by way of the displacement pick-up 55′, such that the distance of the focal plane of the imaging optics corresponds to the distance of the loop 10′ to the distal end of the imaging optics, here the liquid lens 75′, and the loop 10′ is always imaged onto the picture sensor 85′ in a focused manner by way of the liquid lens 75′.

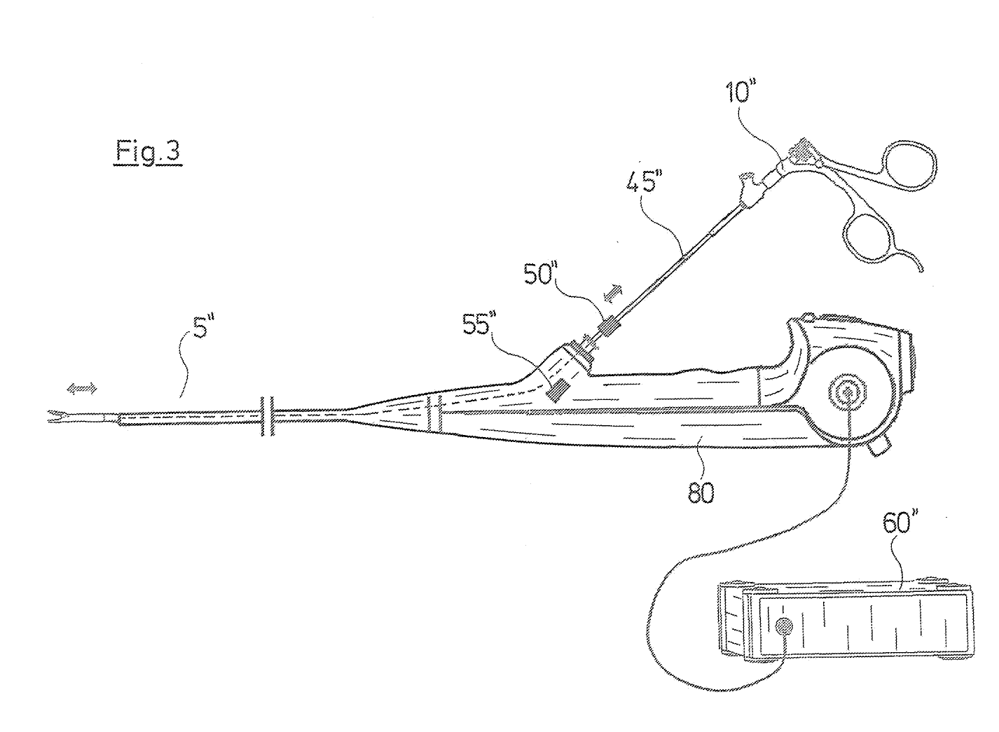

With the endoscope system 5″ represented in FIG. 3, the working instrument and imaging optics (not shown) of the endoscope system 5″ are led in a common endoscope shank (not shown). With the endoscope system 5″ the working instrument, here a forceps 10″, is displaced along the endoscope shank by way of a flexible actuator 45″ which is led in the endoscope shank. The displacement of the forceps 10″ is detected by way of a displacement transmitter 50″ arranged in a fixed manner with regard to the actuator 45″, and a displacement pick-up 55′ which is a arranged in a fixed manner on the handle 80″, and is transferred by a displacement pick-up 55′ to a control device 60″ via a signal corresponding to the displacement of the forceps 10″. With the imaging optics of the endoscope system 5″, a liquid lens with an electrical influencable focal width which forms the objective of the imaging optics of the endoscope system 5″, is set by the control device 60″. The setting thereby is effected as with the previous embodiment examples, in a manner such that the focal width of the liquid lens is changed to such an extent, that the working distance of the imaging optics of the endoscope system 5″ is corrected by the displacement of the forceps 10″ along the optical axis of the imaging optics of the endoscope system 5″.

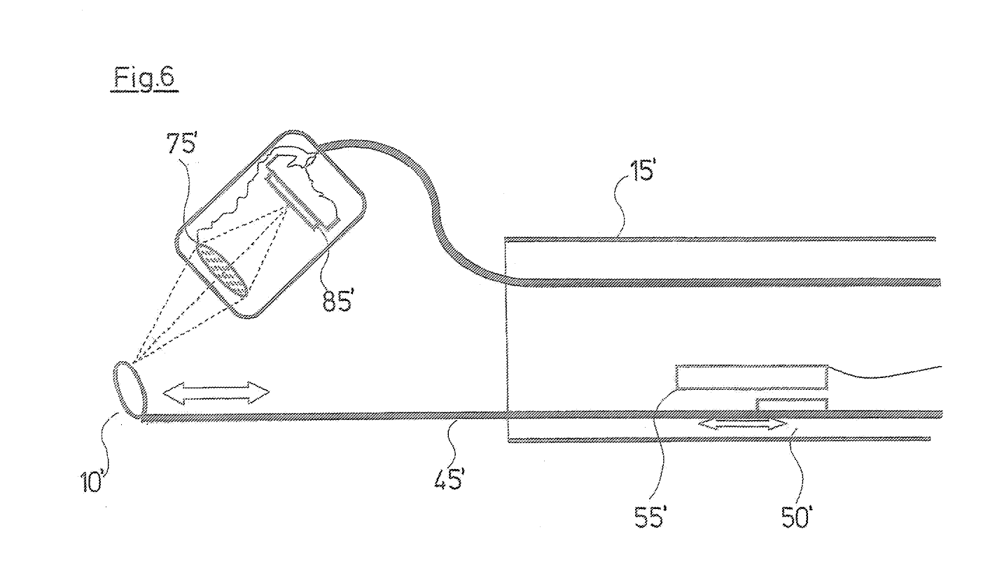

FIG. 6 shows a preferred embodiment, in which the imaging optics consisting of the picture sensor 85′ and lens 75′ are arranged angled to the movement direction of the working instrument or of the actuator 45′. For example, the movement direction of the actuator or working instrument runs at an angle to the optical axis which is defined by the arrangement of the picture sensor 85′ and the objective or lens 75. By way of adapting the shape of the lens designed as a liquid lens 75′, the focal plane may be set such that the loop 10′, which here represents the distal end of the working instrument, is situated in the region of the focal plane. Thereby, the focal plane indeed is not moved by the same amount as the actuator 45′, but by an amount which takes into account the angle between the optical axis and the movement axis of the actuator 45′.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An endoscope system with imaging optics comprising:
at least one adjustable optical element (75, 75′);
a working instrument (10, 10′, 10″) positionable relative to the imaging optics;
a displacement measuring device (50, 50′, 50″, 55, 55′, 55″) designed for detecting a distance covered by the working instrument (10, 10′, 10″) with its positioning; and
a control device (60, 60′, 60″) which adjusts the at least one adjustable optical element (75, 75′) in dependence on a detected displacement.

2. The endoscope system according to claim 1, wherein the imaging optics are arranged with the working instrument (10, 10′, 10″) in a common shank (15, 15′).

3. The endoscope system according to claim 2, wherein the imaging optics are arranged at an angular offset to the working instrument (10, 10′, 10″).

4. The endoscope system according to claim 1, wherein the control device (60, 60′, 60″) is designed for adjusting the at least one adjustable optical element (75, 75′) such that a distance of a focal plane or a position of the imaging optics is changed by the detected displacement of the working instrument (10, 10′, 10″).

5. The endoscope system according to claim 1, wherein the at least one adjustable optical element (75, 75′) is an electromagnetically or piezoelectrically displaceable or electrically changeable liquid lens.

6. The endoscope system according to claim 1, wherein the working instrument (10, 10′, 10″) is selected from the group consisting of a HF-(resection) electrode, a cutting loop, a laser fiber, a lithotripsy probe, gripper forceps, a motor-driven working instrument (10, 10′, 10″), a cutter and a measurement probe.

7. The endoscope system according to claim 1, wherein the displacement measuring device (50, 50′, 50″, 55, 55′, 55″) is designed for contact-free detection of the distance covered by the working instrument (10, 10′, 10″).

8. The endoscope system according to claim 1, wherein the displacement measuring device (50, 50′, 50″, 55, 55′, 55″) comprises:
a displacement pick-up (55′) arranged in a fixed manner with regard to the imaging optics, and a displacement transmitter (50′) which corresponds to the displacement pick-up (55′) and is arranged in a fixed manner with regard to the working instrument (10′); or
a displacement pick-up (55, 55″) arranged in a fixed manner with regard to the working instrument (10, 10″) and a corresponding displacement transmitter (50, 50″) which corresponds to the displacement pick-up (55, 55″) and which is arranged in a fixed manner with regard to the imaging optics.

9. The endoscope system according to claim 1, wherein the displacement measuring device (50, 50′, 50″, 55, 55′, 55″) is a magnet encoder or a plunger coil.

10. The endoscope system according to claim 8, wherein the control device (60, 60′, 60″) is designed for setting the at least one adjustable optical element (75′) in dependence on a prepositioning(s) of the working instrument (10, 10′, 10″) or of the displacement pick-up (55, 55′, 55″) or of the displacement transmitter (50, 50′, 50″).

11. A working instrument (10, 10′, 10″) designed for incorporating into an endoscope system with imaging optics,
the endoscope system comprising:
at least one adjustable optical element (75, 75′);
a working instrument (10, 10′, 10″) positionable relative to the imaging optics;
a displacement measuring device (50, 50′, 50″, 55, 55′, 55″) designed for detecting a distance covered by the working instrument (10, 10′, 10″) with its positioning; and
a control device (60, 60′, 60″) which adjusts the at least one adjustable optical element (75, 75′) in dependence on a detected displacement, and
the working instrument including a displacement pick-up up (55, 55′, 55″) or a displacement transmitter (50, 50′, 50″).
